(19) United States
(12) Patent Application Publication
(10) Pub. No.: US 2024/0355994 A1
(43) Pub. Date: Oct. 24, 2024

(54) APPARATUS AND METHOD FOR SELECTING CATHODE MATERIAL FOR SODIUM-ION BATTERY USING MACHINE LEARNING

(71) Applicant: FOUNDATION OF SOONGSIL UNIVERSITY-INDUSTRY COOPERATION, Seoul (KR)

(72) Inventors: Kyoung Min MIN, Suwon-si (KR); Min Seon KIM, Ansan-si (KR)

(21) Appl. No.: 18/347,691

(22) Filed: Jul. 6, 2023

(30) Foreign Application Priority Data
Apr. 24, 2023 (KR) 10-2023-0053382

(51) Int. Cl. H01M 4/04; H01M 4/525; H01M 10/054

(52) U.S. Cl. CPC H01M 4/04; H01M 4/525; H01M 10/054; H01M 2004/028

(57) ABSTRACT

An apparatus for selecting a sodium-ion battery cathode material using machine learning includes an input data generation unit configured to select candidate materials among a plurality of materials possible to be used as cathode materials for sodium-ion batteries and generate O3 input data and P3 input data respectively for O3 structure materials and P3 structure materials formed depending on structural transition during charge and discharge from each candidate material, and a material classification unit configured to receive the O3 and P3 input data from the input data generation unit and classify the candidate materials depending on stability in a pristine state and desodiated state, respectively, by performing machine learning on data of the plurality of O3 and P3 structure materials using a pristine model and a desodiated model as prediction models.

### CROSS REFERENCE TO RELATED APPLICATION

The present application claims priority to Korean Patent Application No. 10-2023-0053382, filed Apr. 24, 2023, the entire contents of which is incorporated herein for all purposes by this reference.

### BACKGROUND OF THE INVENTION

### Field of the Invention

The present invention relates to an apparatus and method for selecting cathode materials for sodium-ion batteries using machine learning, and more particularly, to a technology of utilizing machine learning instead of experiment to perform material exploration for selecting stable substances available for cathode materials in sodium-ion batteries.

### Description of the Related Art

Currently, lithium-ion batteries (LIBs) are widely used in various fields due to their high energy density and long lifespan. However, the rising cost and scarcity of lithium metal have prompted the necessity for alternative secondary batteries. Sodium-ion batteries (SIBs) are gaining significant attention as a promising candidate to replace lithium-ion batteries as a secondary battery, and research and development in this regard are actively underway.

Sodium-ion batteries, which implement electrochemical mechanisms similar to lithium-ion batteries, leverage the abundant resource of sodium (Na) and allow for easy transition metal insertion, allowing for fast charging speeds. In addition, sodium-ion batteries also possess the advantage of operating over a wide range of temperatures.

The cathode, which is a crucial component of sodium-ion batteries, plays a significant role in determining the battery's capacity, accounting for approximately 35% of the battery's cost, and the development of cathode materials with high performance and stability is considered a key aspect of research on next-generation energy storage devices.

Korean Patent Publication No. 10-2022-0109337 (Title: Electrochemically inactive element substituted electrode active material for sodium secondary battery and sodium secondary battery comprising the same) discloses an electrode active material for sodium secondary batteries, formed by the chemical formula Nay[(Ni₁-aMa)xTM₁₋ₓ]O₂, where M represents Mg, TM is at least one selected from manganese (Mn), titanium (Ti), zirconium (Zr), and tin (Sn), and the conditions of 0.7≤ y≤1, 0.01≤ a≤0.5, and 0.3≤x≤0.7 are specified.

### Documents of Related Art

(Patent Document 1) Korean Patent Publication No. 10-2022-0109337

### SUMMARY OF THE INVENTION

The present invention has been conceived to accomplish the above object through material exploration for selecting stable substances available as cathode materials for sodium-ion batteries using machine learning instead of experiments.

The technical objects of the present invention are not limited to the aforesaid, and other objects not described herein with can be clearly understood by those skilled in the art from the descriptions below.

In order to accomplish the above objects, the present invention includes an input data generation unit configured to select candidate materials among a plurality of materials possible to be used as cathode materials for sodium-ion batteries and generate O3 input data and P3 input data respectively for O3 structure materials and P3 structure materials formed depending on structural transition during charge and discharge from each candidate material, a material classification unit configured to receive the O3 and P3 input data from the input data generation unit and classify the candidate materials depending on stability in a pristine state and desodiated state, respectively, by performing machine learning on data of the plurality of O3 and P3 structure materials using a pristine model and a desodiated model as prediction models, a data sampling unit configured to receive data from the material classification unit and perform data sampling to solve data imbalance between stable and unstable candidate materials in the pristine and desodiated states, respectively, and a selection unit configured to receive data from the data sampling unit and selecting a stable material maintaining stable structure during the charge and discharge of sodium-ion batteries among the candidate materials.

In an embodiment of the present invention, the input data generation unit may perform density functional theory (DFT) calculations on the plurality of possible materials to obtain an energy difference value (ED) between the O3 structure material and the P3 structure material.

In an embodiment of the present invention, the possible materials may be represented by a formula NaₓNi₁D^{a}yD^{b}_{z}O₂.

In an embodiment of the present invention, Da and Db may each be one element selected from the group consisting of Zr, Se, Fe, Zn, Sc, Cu, Y, Sb, Cr, W, Nb, Co, V, Mo, B, Ti, Mn, As, Te, Mg, Al, Ta, La, Sn, Ge, Si, and Ga.

In an embodiment of the present invention, the input data generation unit may select the candidate materials by excluding materials unable to achieve structural stabilization among the plurality of possible materials.

In an embodiment of the present invention, the material classification unit generates the pristine and desodiated models by training a classification model.

In an embodiment of the present invention, the classification model may be one of Extra Trees Classifier model, Random Forest model, K-Nearest Neighbors Classifier model, Light Gradient Boosting Machine (LightGBM) model, and Logistic Regression model.

In an embodiment of the present invention, the data sampling unit may perform oversampling and undersampling sequentially for the data sampling.

In an embodiment of the present invention, the data sampling unit may use Synthetic Minority Oversampling Technique (SMOTE) for performing the oversampling.

In an embodiment of the present invention, the data sampling unit may use Tomek Links and Edited Nearest Neighbors (ENN) for performing the undersampling.

In order to accomplish the above objects, the present invention includes the first step of generating, by the input data generation unit, the O3 and P3 input data, the second step of classifying, by the material classification unit, the candidate materials depending on stability in the pristine state and desodiated state, respectively, generated by training a classification model, the third step of performing, by the data sampling unit, oversampling and undersampling sequentially to solve data imbalance between the stable and unstable candidate materials in the pristine and desodiated states, respectively, and the fourth step of deriving, by the selection unit, the stable material by selecting the candidate material stable in both the pristine and desodiated stages.

### BRIEF DESCRIPTION OF THE DRAWINGS

FIG. 1 is a diagram illustrating the principle of a sodium-ion battery;

FIGS. 2 and 3 are diagrams illustrating the structural transitions in the cathode material of a sodium-ion battery according to charge and discharge in an embodiment of the present invention.

FIG. 4 is a block diagram illustrating a configuration of a cathode material selection apparatus according to an embodiment of the present invention;

FIG. 5 is a table of input features of an input data generation unit according to an embodiment of the present invention;

FIG. 6 is a table of the use of classification models according to an embodiment of the present invention;

FIG. 7 is a graph illustrating the distribution of candidate materials based on stability according to an embodiment of the present invention;

FIGS. 8 to 11 are diagrams illustrating a data sampling process in a data sampling unit according to an embodiment of the present invention;

FIGS. 12 to 15 are diagrams and related graphs illustrating a material selection process in a cathode material selection unit according to an embodiment of the present invention;

FIGS. 16 and 17 are graphs and tables related to the performance of a cathode material selection unit according to an embodiment of the present invention; and

FIG. 18 is a table of the materials selected by a selection unit according to an embodiment of the present invention.

### DETAILED DESCRIPTION OF THE INVENTION

Hereinafter, the present invention will be described with reference to the accompanying drawings. However, the present invention may be embodied in many different forms and is not limited to the embodiments described herein. In order to clearly describe the present invention, parts irrelevant to the description may be omitted in the drawings, and similar reference numerals may be used for similar components throughout the specification.

Throughout the specification, when a part is said to be “connected (coupled, contacted, or combined)” with another part, this is not only “directly connected”, but also “indirectly connected” with another member in between. Also, when a part is said to “comprise” a certain component, this means that other components may be further included instead of excluding other components unless specifically stated otherwise.

The terminology used herein is for the purpose of describing particular embodiments only and is not intended to be limiting of the invention. As used herein, the singular forms are intended to include the plural forms as well, unless the context clearly indicates otherwise. It will be further understood that the terms “comprises” or “has,” when used in this specification, specify the presence of a stated feature, number, step, operation, component, element, or a combination thereof, but they do not preclude the presence or addition of one or more other features, numbers, steps, operations, components, elements, or combinations thereof.

Hereinafter, the present invention will be described in detail with reference to the accompanying drawings.

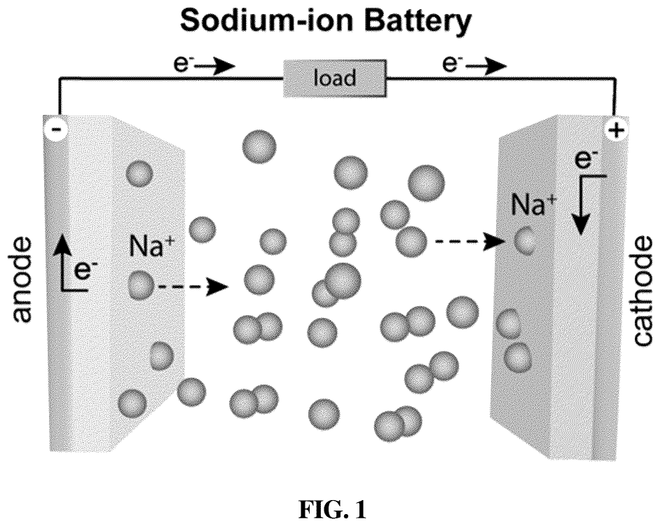

FIG. 1 is a diagram illustrating the principle of a sodium-ion battery. As shown in FIG. 1, charging and discharging occur through the movement of sodium ions (Na+) and electrons (e−) and, in this process, the stability of the cathode material plays a crucial role in enhancing the capacity and overall performance of the sodium-ion battery.

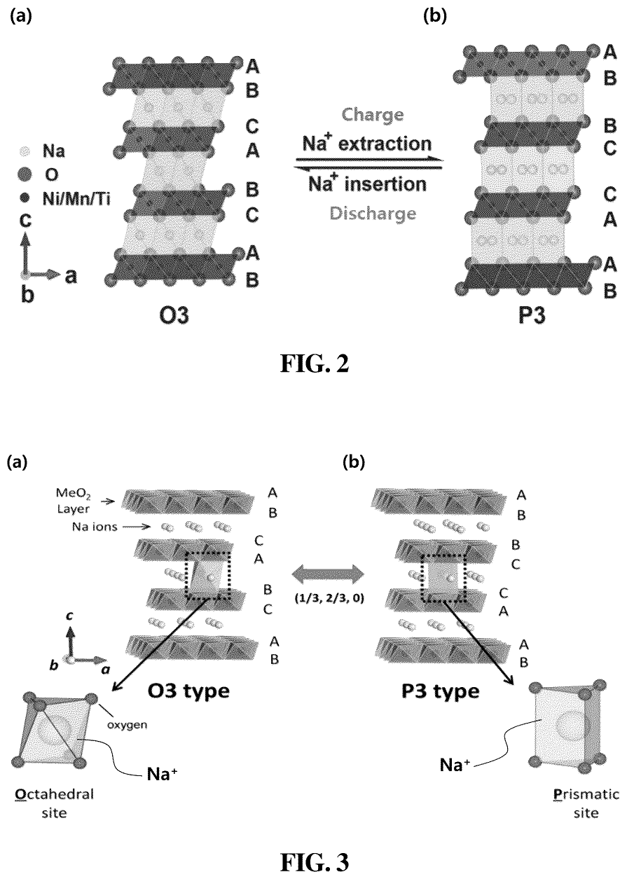

FIGS. 2 and 3 are diagrams illustrating the structural transitions in the cathode material of a sodium-ion battery according to charge and discharge in an embodiment of the present invention. Layered transition metal oxide compounds (LTMOs) are utilized as cathode materials in sodium-ion batteries, and part (a) of FIG. 2 represents the O3 structure of the cathode material in its charged or discharged state, while part (b) of FIG. 2 represents the P3 structure of the cathode material in the charged state.

Additionally, part (a) of FIG. 3 shows the cathode material structure as the O3 structure and the corresponding sodium ion (Na+) arrangement within that structure, while part (b) of FIG. 3 shows the cathode material structure as the P3 structure and the corresponding sodium ion (Na+) arrangement within that structure.

As shown in FIGS. 2 and 3, in the cathode material with an O3 structure, sodium ions (Na+) reside in the Octahedral sites, forming a repeating ABC layer structure, while, in the cathode material with a P3 structure, sodium ions (Na+) occupy the Prismatic sites, resulting in a repeating AB-BC layer configuration.

Layered transition metal oxides offer the advantages of excellent Na-ion storage performance, allowing for the realization of high-energy density and high-capacity batteries, while also facilitating easy material synthesis due to the abundance of sodium.

However, as shown in FIGS. 2 and 3, in layered transition metal oxides, an irreversible transformation occurs in the cathode material structure between O3 and P3 during charging and discharging, which may lead to a decrease in sodium-ion battery capacity and stability. Therefore, it is necessary to develop cathode materials that can suppress such structural phase transitions or allow reversible phase transitions in order to mitigate the detrimental effects caused by them.

However, conducting experiments with all potentially viable materials as cathode materials would be time-consuming and costly, so the present invention proposes an apparatus and method utilizing machine learning for material design.

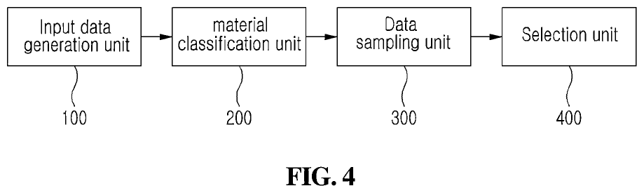

FIG. 4 is a block diagram illustrating a configuration of a cathode material selection apparatus according to an embodiment of the present invention. As shown in FIG. 4, a cathode material selection apparatus of the present invention includes an input data generation unit 100 that selects candidate materials among a plurality of possible materials available as cathode material for sodium-ion batteries and generates, per candidate material, O3 input data and P3 input data respectively for O3 structure materials and P3 structure materials formed based on the structural transitions during charging and discharging, a material classification unit 200 that receives the O3 input data and P3 input data from the input data generation unit and performs machine learning on the data of multiple O3 structure materials and multiple P3 structure materials using the predictive models, namely the Pristine Model and the Desodiated Model to classify candidate materials based on their stability respectively in the pristine state and desodiated state, a data sampling unit 300 that receives data from the material classification unit 200 and performs data sampling to mitigate the data imbalance between stable candidate materials and unstable candidate materials in both the pristine state and desodiated state during, and a selection unit 400 that receives data from the data sampling unit 300 and selects stable materials, which maintain a stable structure during the charge and discharge of sodium-ion batteries, among the candidate materials.

The input data generation unit 100, classification unit 200, data sampling unit 300, and selection unit 400 may each include hardware units such as computers or central processing units (CPUs), software units such as computational programs, and units implemented by a combination of both hardware and software. In addition, one unit may be implemented using two or more hardware components, and two or more units may also be implemented using a single hardware component.

The machine learning-based sodium-ion battery cathode material selection apparatus is characterized by the chemical formula NaₓNi₁D^{a}_{y}D^{b}_{z}O₂ represent the possible materials. (where D^{a} and D_{b} are arbitrary elements, and 0.5≤x≤1, and y:z=0.25:0.25 or 0.42:0.08 or 0:0.5)

In this case, D^{a} and D^{b} may each be any one element selected from the group consisting of Zr, Sc. Fc, Zn, Sc, Cu, Y, Sb, Cr, W, Nb, Co, V, Mo, B, Ti, Mn, As, Te, Mg. Al, Ta, La, Sn, Ge, Si, and Ga.

In an embodiment, the input data generation unit 100 may store information about elements widely used as dopants in the cathode and information about the chemical formulas of possible materials that can be used as cathodes, using the aforementioned 27 elements.

In the input data generation unit 100, it is possible to generate a plurality of possible materials by applying the aforementioned elements to the positions of dopant elements (D^{a} and D^{b}) in the chemical formulas of the aforementioned possible materials. In detail, by applying the aforementioned 27 elements, it is possible to generate 1,458 materials, and by generating data for O3 structure materials and P3 structure materials for each material, data for 2,916 materials can be generated.

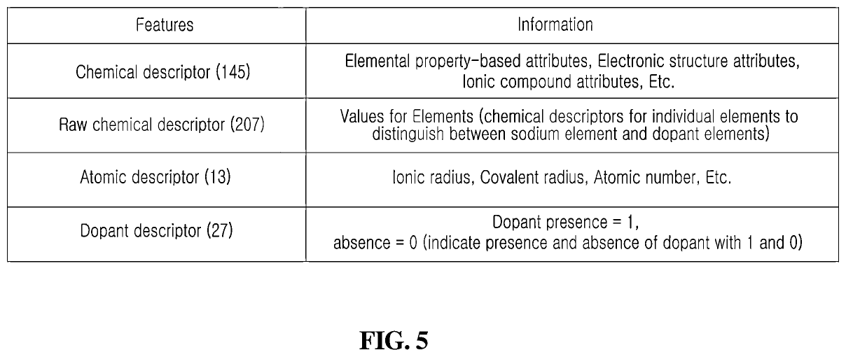

FIG. 5 is a table of input features of the input data generation unit 100 according to an embodiment of the present invention.

The input data generation unit 100 is capable of performing density functional theory (DFT) calculations for a plurality of candidate materials to compute the energy difference value (ED) between O3 structure materials and P3 structure materials. Furthermore, the input data generation unit 100 may exclude materials that cannot be structurally stabilized among the plurality of candidate materials.

In detail, the input data generation unit 100 may compute the structural energy for each of the 2,916 materials using density functional theory and set the ED value as the difference between the structural energies of the corresponding O3 structure material and the P3 structure material for each candidate material.

The input data generation unit 100 may identify materials with positive ED values as materials that cannot be structurally stabilized and exclude them from the plurality of possible materials to generate the candidate material data.

As an example, out of the 2,916 material data (related to a total of 1,458 possible materials), 14 material data (related to 7 possible materials) may be excluded, resulting in the selection of 2,902 candidate materials (related to 1,451 possible materials).

Additionally, input data for the O3 structure material, called O3 input data, and input data for the P3 structure material, called P3 input data, may be generated for these candidate materials.

The O3 input data may include data on chemical descriptors, raw chemical descriptors, atomic descriptors, and dopant descriptors as input features.

Similarly, the P3 input data may include data on chemical descriptors, raw chemical descriptors, atomic descriptors, and dopant descriptors as input features.

Here, as the features related to the chemical descriptors, information such as elemental property-based attributes, ionic compound attributes, and electronic structure attributes may be included.

Moreover, as the features related to the raw chemical descriptors, information about the structural energy values of the O3 and P3 structure materials, and they are chemical descriptors for individual elements to distinguish between the sodium element and dopant elements. Particularly, as features related to the raw chemical descriptors, information about O3 and P3 structures, when the ions (sodium ions) are immobile, in each candidate material and structural energies of the respective structures and information about O3 and P3 structures, when the ions (sodium ions) are mobile, in each candidate material and structural energies of the respective structures.

Additionally, as features related to the atomic descriptors, all information related to the elements included in the candidate materials, such as ionic radius, covalent radius, and atomic number, may be included.

Furthermore, in the dopant descriptors, one-hot encoding is performed as a preprocessing step in machine learning, generating data that indicates the presence or absence of each of the 27 elements, which may also be used as input features. Thus, the number of input features may increase according to the number of dopant elements (e.g., 27 in this specific embodiment).

The O3 input data and P3 input data are passed to the material classification unit 200, which may use machine learning to perform training on the O3 input data and P3 input data separately and classify them into stable and unstable candidate materials based on stability.

Using the training data, the classification model may generate two prediction models: the pristine model and the desodiated model.

In this case, the classification model may be selected from the Extra Trees Classifier model, Random Forest model, K-Nearest Neighbors Classifier model, light gradient Boosting Machine (LightGBM) model, and Logistic Regression model.

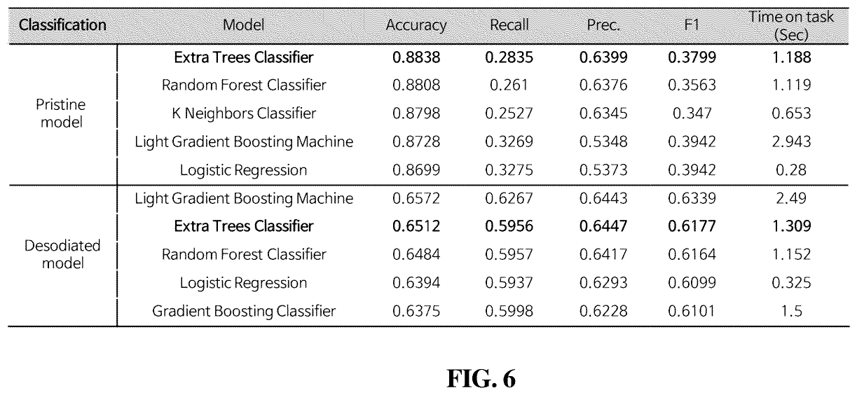

However, in the table of the use of classification models as shown in FIG. 6 according to an embodiment of the present invention, the Extra Trees Classifier model is preferably used for classification, and it demonstrates superior classification performance when used for the Pristine Model and Desodiated Model.

Here, the pristine model may be obtained by training a classification model using the training data, and in this case, the material classification unit 200 selects data of O3 structure materials and P3 structure materials in a state without ion movement, specifically the Pristine state, from the O3 input data and P3 input data as training data and utilizes this training data to train the classification model, generating the pristine model.

Additionally, the desodiated model may also be obtained by training a classification model using the training data, and in this case, the material classification unit 200 selects data of O3 structure materials and P3 structure materials in a state with ion movement, specifically the desodiated state, from the O3 input data and P3 input data as training data and utilizes this training data to train the classification model, generating the desodiated model.

The material classification unit 200 may generate prediction models for the pristine model and the desodiated model using the training data as described above, and classify the candidate materials based on stability in the respective pristine and desodiated states by inputting the O3 input data and P3 input data into the pristine model and the desodiated model.

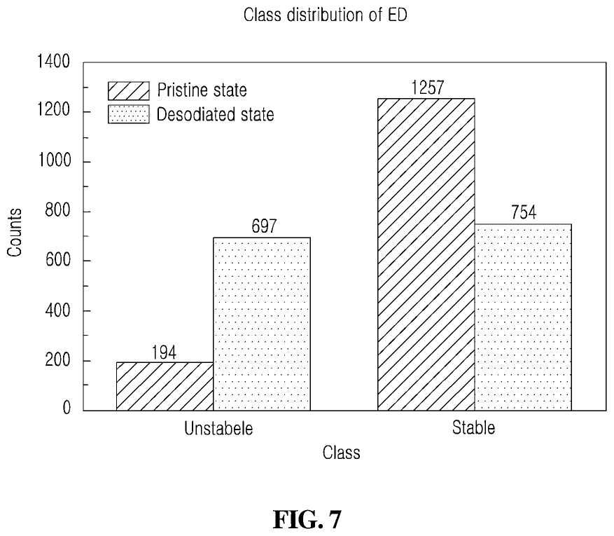

FIG. 7 is a graph illustrating the distribution of candidate materials based on stability according to an embodiment of the present invention. In FIG. 7, the horizontal axis may represent the classification of each candidate material into stable and unstable states in the pristine model and the desodiated model, respectively, and the vertical axis may represent the counts of candidate materials.

Here, stability refers to a negative value of the ED value, and in the pristine model, the ED values of candidate materials may be derived using O3 input data and P3 input data to classify them into stable and unstable candidates, and similarly in the desodiated model, the ED values of candidate materials may be derived using O3 input data and P3 input data to classify them into stable and unstable candidates.

As a specific embodiment shown in FIG. 7, the classification was performed for the total of 1,451 candidate materials using the pristine model and desodiated model, where the pristine model classified 1,257 candidates as stable and 194 candidates as unstable in the pristine state, while the desodiated model classified 754 candidates as stable and 697 candidates as unstable in the desodiated state.

However, as shown in FIG. 7, when performing classification for candidate materials using the pristine model and desodiated model respectively, the pristine model shows an imbalanced distribution (approximately 6 times more stable candidates than unstable candidates) while the desodiated model shows a uniform distribution, and such data imbalance may have a detrimental effect on the results of machine learning, necessitating the data sampling unit 300 to perform data sampling to solve this data imbalance problem as described below.

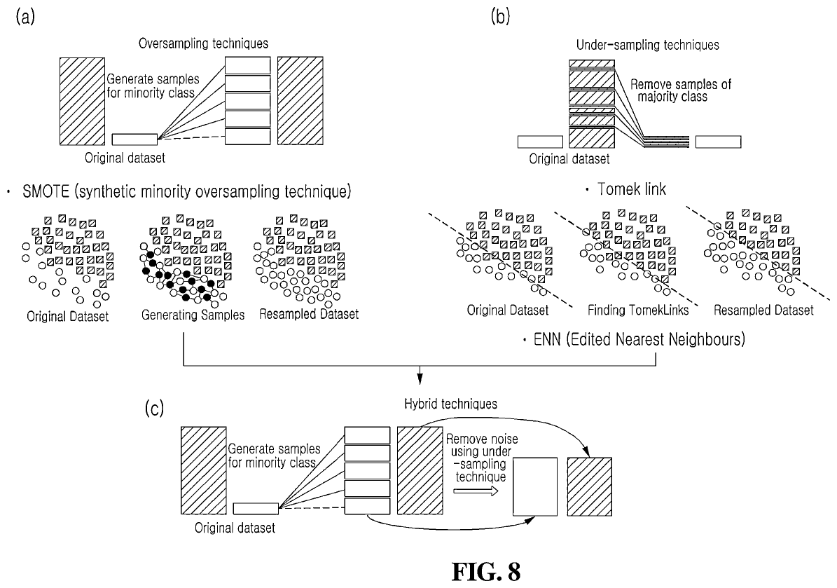

FIGS. 8 to 11 are diagrams illustrating a data sampling process in the data sampling unit 300 according to an embodiment of the present invention. In detail, FIG. 8 illustrates the entire process of data sampling performed by the data sampling unit 300.

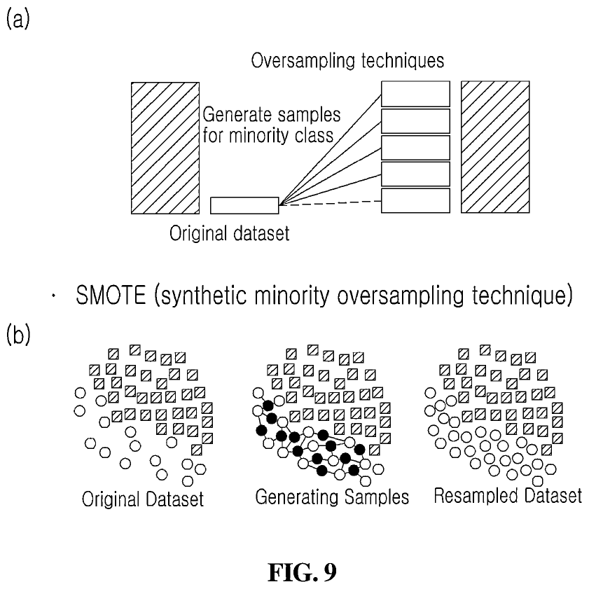

FIG. 9 is an enlarged image of part (a) of FIG. 8, part (a) of FIG. 9 illustrates the process of oversampling, and part (b) of FIG. 9 illustrates the execution process of SMOTE as an oversampling technique.

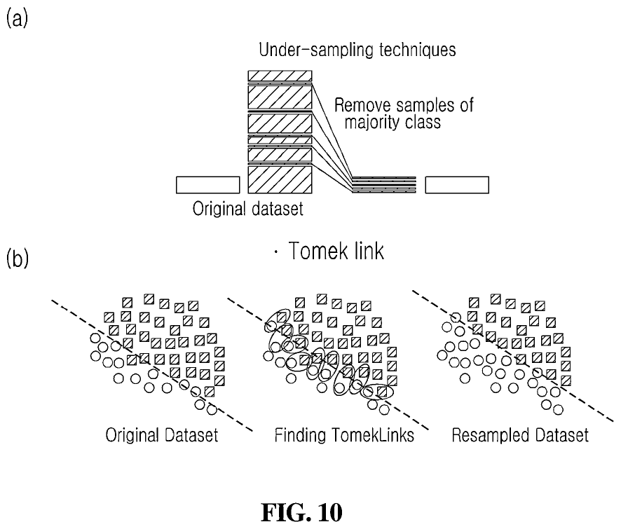

In addition, FIG. 10 is an enlarged image of part (b) of FIG. 8, part (a) of FIG. 10 represents the Tomek Links undersampling model, and part (b) of FIG. 10 represents the ENN undersampling model.

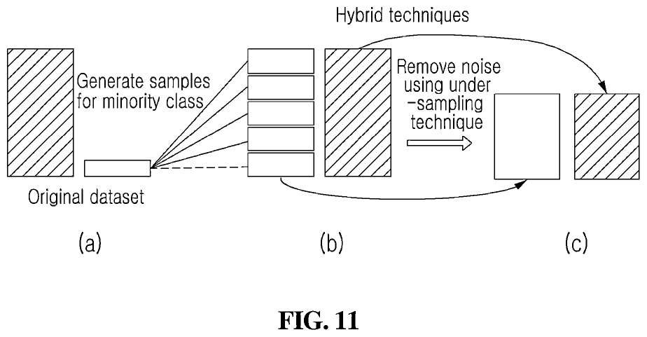

FIG. 11 is an enlarged image of part (c) of FIG. 8. Here, part (a) of FIG. 11 represents the original dataset in an imbalanced state before data sampling, part (b) represents the dataset after oversampling, and part (c) represents the dataset after undersampling.

As shown in FIGS. 8 to 11, the data sampling unit 300 may perform oversampling and undersampling sequentially for data sampling. Here, the data sampling unit 300 may utilize Synthetic Minority Oversampling Technique (SMOTE) for oversampling. For undersampling, the data sampling unit 300 may utilize Tomek Links and Edited Nearest Neighbors (ENN). SMOTE is a data augmentation technique, while Tomek Links and ENN are methods for removing data interfering with predictions.

SMOTE utilizes the k-nearest neighbors (k-NN) algorithm on the original data to artificially increase the data in the same class in such a way as to select k nearest neighbor vectors (samples) among data of a minority class, draw line segments between the original vector and the selected vectors to creating random points on the line segments as new vectors (or arbitrary one among them).

Tomek Links is a method that removes data with higher distribution within the Tomek links, which are formed by connecting the closest data points between different classes, allowing significant prevention of information loss compared to random deletion sampling. ENN is a KNN-based method used to remove data with a high distribution.

The data sampling unit 300 may receive pristine classification data about stable and unstable candidate materials in the pristine state and desodiated classification data about stable and unstable candidate materials in the desodiated state from the material classification unit 200 and perform data sampling on the received data.

As shown in part (a) of FIG. 8 and FIG. 9, firstly the data sampling unit 300 may perform oversampling. In this case, as described above, SMOTE may be used to address the imbalance in data between stable and unstable candidate materials in the pristine classification data and the desodiated classification data.

In detail, in the data sampling of the pristine classification data, it is possible to perform oversampling on the 1,257 samples of stable candidate materials and 194 samples of unstable candidate materials. By increasing the number of data samples for unstable candidate materials, it is possible to equalize the number of data between stable and unstable candidate materials.

Next, by performing undersampling on the data obtained through oversampling, as seen in part (b) of FIG. 8 and FIG. 10, the number of data samples for stable and unstable candidate materials may be reduced, thereby achieving a balanced distribution and reducing computational time, resulting in enhanced machine learning effectiveness.

Here, data sampling for the desodiated classification data may also be performed using the same process as the data sampling for the pristine classification data.

The present invention performs data sampling using a hybrid approach that combines oversampling to augment scarce data and undersampling to reduce abundant data, thereby resolving data imbalance and enhancing the efficiency of machine learning.

The selection unit 400 receives the data that has been subjected to the above-described data sampling, extracts stable candidate materials from the sampled pristine classification data, and identifies materials corresponding to the stable candidate materials in the desodiated classification data, thereby enabling the selection of stable materials that can be used as cathode materials in sodium-ion batteries.

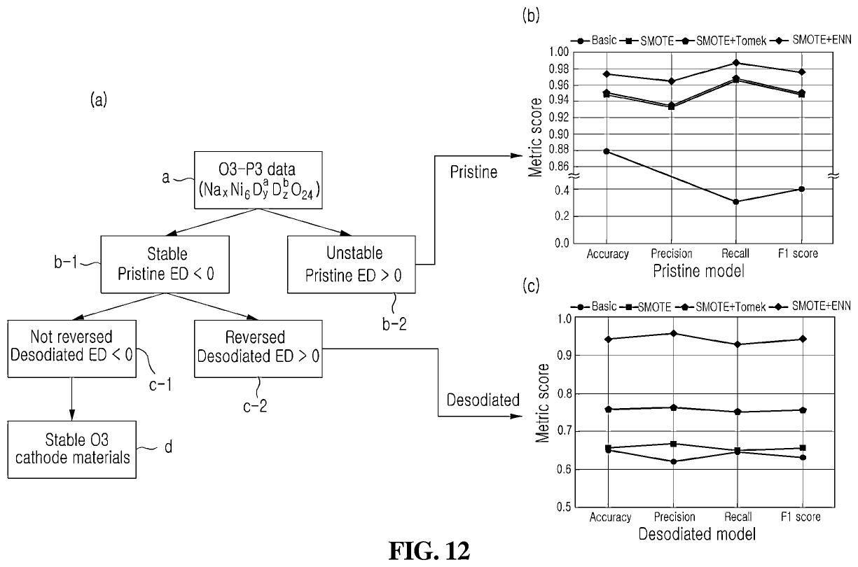

FIGS. 12 to 15 are diagrams and related graphs illustrating a material selection process in a cathode material selection unit according to an embodiment of the present invention. In detail, FIG. 12 shows the processes at the input data generation unit 100, material classification unit 200, data sampling unit 300, and selection unit 400 and graphs representing performances of process models.

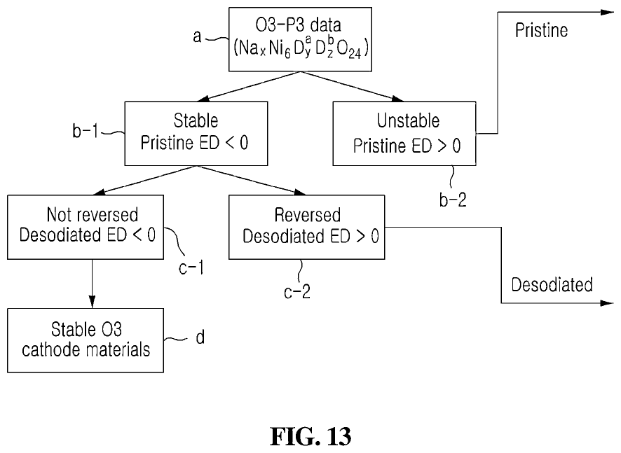

FIG. 13 is an enlarged image of part (a) of FIG. 12. In part (a) of FIG. 12 and FIG. 13, a denotes the process performed by the input data generation unit 100, material classification unit 200, and data sampling unit 300, b-1 and b-2 denote the process in the selection unit 400 for classifying stable candidate materials from the sampled pristine classification data, c-1 and c-2 denote the process in the selection unit 400 for selecting candidate materials that match stable candidate materials from the pristine classification data in the desodiated classification data, and d denotes the process in the selection unit 400 for extracting stable materials that match the candidate materials.

As shown in part (a) of FIG. 12 and FIG. 13, the cathode material selection apparatus of the present invention selects stable candidate materials from the sampled pristine classification data and matches the selected stable candidate materials with stable candidate materials from the sampled desodiated classification data, thereby extracting stable materials present in both the sampled pristine classification data and the sampled desodiated classification data as suitable materials for the cathode of a sodium-ion battery.

That is, the cathode material selection apparatus of the present invention may extract materials with the structure where E(O3)<E(P3) both in the pristine state and desodiated state, allowing for the selection of suitable materials for the cathode. (where E(O3) represents the structural energy of O3 structure materials, and E(P3) represents the structural energy of P3 structure materials.)

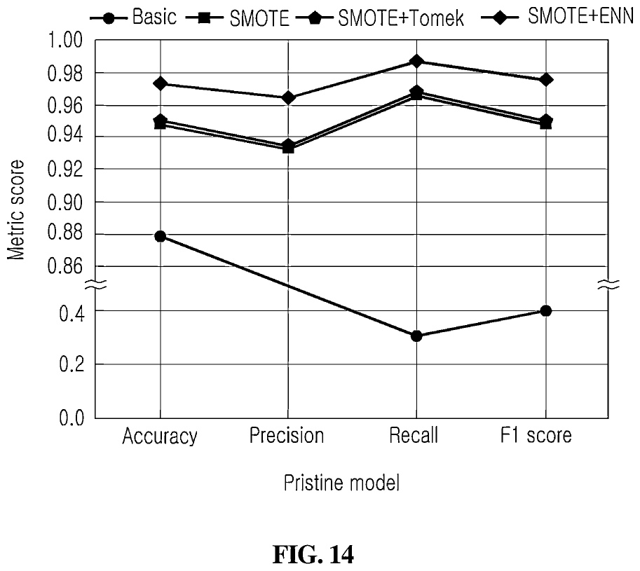

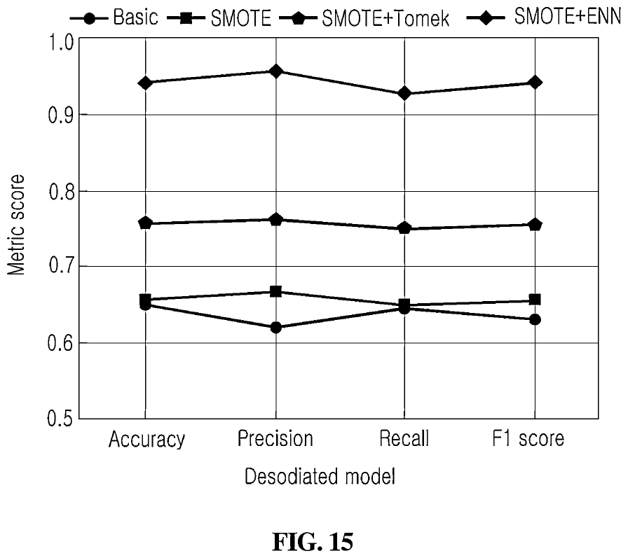

FIG. 14 is an enlarged image of part (b) of FIG. 12, and FIG. 15 is an enlarged image of part (c) of FIG. 12. Here, part (b) of FIG. 12 and FIG. 14 represent graphs comparing the performances of different machine learning models when applying different data sampling techniques to the pristine classification data, while part (c) of FIG. 12 and FIG. 15 represent graphs comparing the performances of different machine learning models when applying different data sampling techniques to the pristine classification data.

In FIGS. 12, 14, and 15, “basic” refers to the performance of the Extra Trees Classifier model alone in the machine learning process, “SMOTE” represents the performance when both the Extra Trees Classifier model and the SMOTE oversampling technique are utilized, “SMOTE+Tomek” represents the performance when the Extra Trees Classifier model, SMOTE oversampling technique, and Tomek Links undersampling technique are combined in the machine learning process, and “SMOTE+ENN” represents the performance when the Extra Trees Classifier model, SMOTE oversampling technique, and ENN undersampling technique are combined in the machine learning process.

As shown in FIGS. 12, 14, and 15, when performing machine learning using data from both the pristine state and the desodiated state, it can be seen that the SMOTE+ENN machine learning model yields the highest scores (Accuracy, Precision, Recall, and F1 Score) in each respective metric for both states, demonstrating that the performance is overwhelmingly superior when using the SMOTE+ENN model.

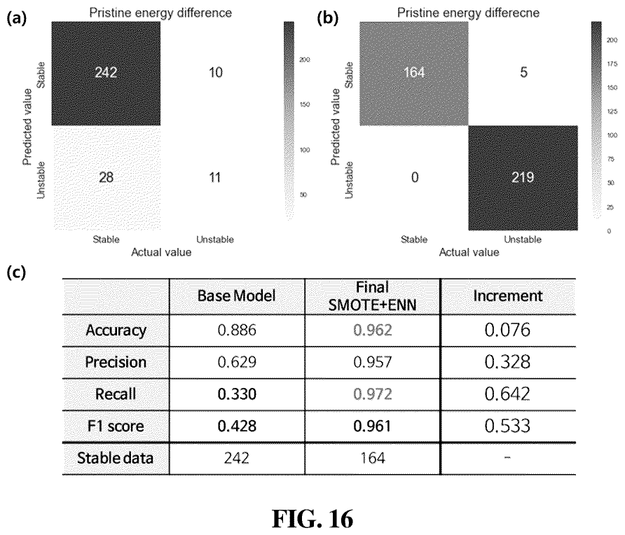

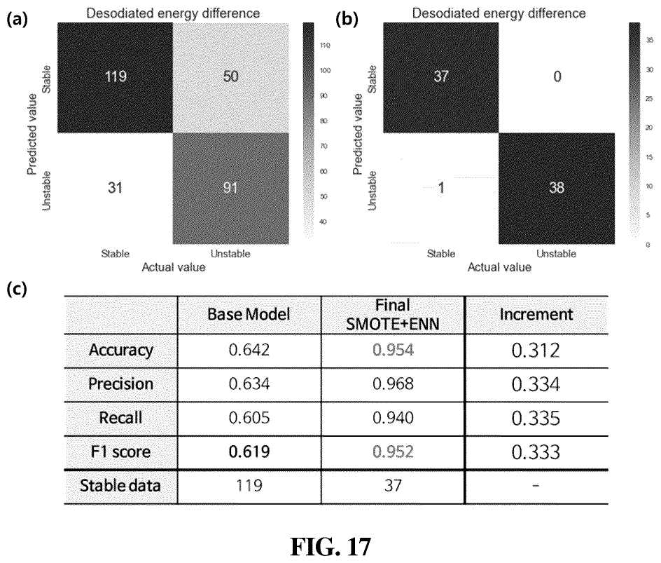

FIGS. 16 and 17 are graphs and tables related to the performance of a cathode material selection apparatus according to an embodiment of the present invention. Here, to assess the performance, ten machine learning models were generated, and their average values were calculated, with a data ratio of 8:2 for training and testing data, respectively, for each model.

Here, FIG. 16 represents the confusion matrix the performances of the machine learning models for candidate materials in the pristine state and a table summarizing the corresponding machine learning models. Meanwhile, FIG. 17 represents the performances of the machine learning models for candidate materials in the desodiated state.

In part (c) of FIG. 16 and part (c) of FIG. 17, “Base Model” refers to a machine learning model that utilizes only the Extra Trees Classifier, while “Final SMOTE+ENN model” refers to a machine learning model that incorporates the Extra Trees Classifier along with the SMOTE oversampling model and the ENN undersampling model.

Part (a) of FIG. 16 and part (a) of FIG. 17 show the confusion matrices for candidate materials in both the pristine and desodiated states when using the Base Model for machine learning, and part (b) of FIG. 16 and part (b) of FIG. 17 show the confusion matrix for candidate materials in both the pristine and desodiated states when using the Final SMOTE+ENN model for machine learning.

It can be observed from FIGS. 16 and 17 that the performance is significantly improved when using the Final SMOTE+ENN model in the cathode material selection apparatus of the present invention, with a notable increase in Recall value for the pristine state machine learning and a significant increase in F1 Score for the desodiated state machine learning, demonstrating a remarkable improvement in performance.

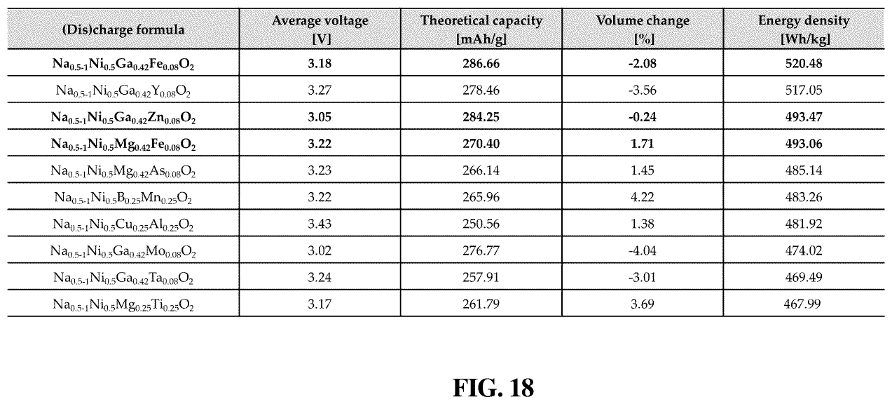

FIG. 18 is a table of the materials selected by the selection unit 400 according to an embodiment of the present invention. FIG. 18 shows a table summarizing the top 10 stable materials with superior average voltage and theoretical capacity among the selected stable materials after undergoing the third screening process to be described hereinbelow.

As described above, the selection unit 400 is capable of identifying stable materials and may subsequently perform analysis on these stable materials. Such additional analysis may be carried out through simulations involving the utilization of stable materials as electrodes to form sodium-ion batteries.

In detail, after performing the first screening process for selecting stable materials as described above, the selection unit 400 may perform a second screening process to identify stable materials capable of achieving an average voltage (average) above a pre-stored threshold voltage.

Furthermore, the selection unit 400 may perform a third screening process to identify stable materials capable of achieving a theoretical electric capacity (theoretical capacity) above a pre-stored capacity threshold in the sodium-ion battery using the stable materials identified during the second screening process.

Furthermore, the selection unit 400 may perform a fourth screening process to identify stable materials capable of achieving implementation by analyzing the volume change of the sodium-ion battery using the stable materials selected in the third screening process and determining whether the volume change rate is below a pre-stored volume change rate threshold, allowing for selecting the corresponding stable materials as applicable materials for final utilization.

In a specific embodiment, the selection unit 400 may potentially screen 637 stable materials through the first screening process, and among them, identify 177 materials as stable materials suitable for utilization as the cathode material in sodium-ion batteries, forming an average voltage above 3V, in the second screening process.

Furthermore, in the third screening process, 155 materials may be identified as stable materials suitable for utilization as the cathode material in sodium-ion batteries, forming a theoretical capacity above 200 mAh/g, and in the fourth screening process, 128 materials may be selected as stable materials suitable for utilization as the cathode material in sodium-ion batteries, forming a volume change rate below 5%, thereby resulting in the derivation of these 128 stable materials as applicable materials.

Hereinafter, a description is made of the cathode material selection method using the cathode material selection apparatus of the present invention.

At the first step, the input data generation unit 100 may generate O3 input data and P3 input data.

An the second step, the material classification unit 200 may train classification models to generate a pristine model and a desodiated model and classify, using the pristine and desodiated models, candidate materials based on their stability in the pristine state and desodiated state, respectively.

At the third step, the data sampling unit 300 may perform oversampling and undersampling sequentially to address the data imbalance of stable and unstable candidate materials in the pristine state and desodiated state, respectively.

After, at the fourth step, the selection unit 400 may screen stable candidate materials in both the pristine and desodiated states to derive stable materials. At the fifth step, the selection unit 400 may perform simulations on sodium-ion batteries using the stable materials to compute and derive the average voltage, theoretical capacity, and volume change rate of the batteries, and during this process, the selection unit 400 may also perform the second, third, and fourth screening processes as described above.

The remaining detailed aspects of the cathode material selection method of the present invention are the same as the disclosed details of the cathode material selection apparatus described above.

By utilizing the cathode material selection apparatus and method of the present invention with the above-described configuration, it is possible to assess the suitability of a plurality of materials as cathode materials for sodium-ion batteries without the need for separate experiments, which allows for a reduction in the time required for the screening of potential cathode materials for sodium-ion batteries, improving the efficiency of sodium-ion battery development.

Furthermore, by utilizing a hybrid approach incorporating both oversampling and undersampling techniques during the machine learning process, it is possible to reduce the screening time using machine learning and improve the precision of cathode material selection.

The advantageous effect of the present invention, based on the above-configuration, is the ability to assess the suitability of multiple materials as cathode materials for sodium-ion batteries without the need for separate experiments, thereby reducing the time for selecting potential cathode materials and enhancing the efficiency of sodium-ion battery development.

Furthermore, the advantageous effect of the present invention lies in utilizing a hybrid approach that combines oversampling and undersampling during machine learning, thereby reducing the screening time using machine learning and improving the precision of cathode material selection.

It should be understood that the advantages of the present invention are not limited to the aforesaid but include all advantages that can be inferred from the detailed description of the present invention or the configuration specified in the claims.

The above description of the present invention is for illustrative purposes only, and it will be understood by those skilled in the art that various modifications and changes can be made thereto without departing from the spirit and scope of the invention. Therefore, it should be understood that the embodiments described above are exemplary and not limited in all respects. For example, each component described as a single type may be implemented in a distributed manner, and similarly, components described as distributed may be implemented in a combined form.

The scope of the invention should be determined by the appended claims, and all changes or modifications derived from the meaning and scope of the claims and equivalent concepts thereof should be construed as being included in the scope of the present invention.

### DESCRIPTION OF REFERENCE NUMERALS

100: input data generation unit
200: material classification unit
300: data sampling unit
400: selection unit

1. An apparatus for selecting a sodium-ion battery cathode material using machine learning, the apparatus comprising at least one processor including:
an input data generation unit configured to select candidate materials among a plurality of possible materials to be used as cathode materials for sodium-ion batteries and generate O3 input data and P3 input data respectively for O3 structure materials and P3 structure materials formed depending on structural transition during charge and discharge from each candidate material;
a material classification unit configured to receive the O3 and P3 input data from the input data generation unit and classify the candidate materials depending on stability in a pristine state and desodiated state, respectively, by performing machine learning on data of the plurality of O3 and P3 structure materials using a pristine model and a desodiated model as prediction models;
a data sampling unit configured to receive data from the material classification unit and perform data sampling to solve data imbalance between stable and unstable candidate materials in the pristine and desodiated states, respectively; and
a selection unit configured to receive data from the data sampling unit and selecting a stable material maintaining stable structure during the charge and discharge of sodium-ion batteries among the candidate materials.

2. The apparatus of claim 1, wherein the input data generation unit performs density functional theory (DFT) calculations on the plurality of possible materials to obtain an energy difference value (ED) between the O3 structure material and the P3 structure material.

3. The apparatus of claim 1, wherein the plurality of possible materials are represented by a formula NaₓNi₁D^{a}_{y}D^{b}_{z}O₂ (where D^{a} and D^{b} are arbitrary elements, 0.5≤x≤1, and y:z=0.25:0.25, 0.42:0.08, or 0:0.5).

4. The apparatus of claim 1, wherein each of D^{a} and D^{b} is at least one element selected from the group consisting of Zr, Se, Fe, Zn, Sc, Cu, Y, Sb, Cr, W, Nb, Co, V, Mo, B, Ti, Mn, As, Te, Mg, Al, Ta, La, Sn, Ge, Si, and Ga.

5. The apparatus of claim 1, wherein the input data generation unit selects the candidate materials by excluding materials unable to achieve structural stabilization among the plurality of possible materials.

6. The apparatus of claim 1, wherein the material classification unit generates the pristine and desodiated models by training a classification model.

7. The apparatus of claim 6, wherein the classification model is one of Extra Trees Classifier model, Random Forest model, K-Nearest Neighbors Classifier model, Light Gradient Boosting Machine (LightGBM) model, and Logistic Regression model.

8. The apparatus of claim 1, wherein the data sampling unit performs oversampling and undersampling sequentially for the data sampling.

9. The apparatus of claim 8, wherein the data sampling unit uses Synthetic Minority Oversampling Technique (SMOTE) for performing the oversampling.

10. The apparatus of claim 8, wherein the data sampling unit uses Tomek Links and Edited Nearest Neighbors (ENN) for performing the undersampling.

11. A method for selecting a sodium-ion battery cathode material using the apparatus of claim 1, the method comprising:
generating, by the input data generation unit, the O3 and P3 input data;
classifying, by the material classification unit, the candidate materials depending on stability in the pristine state and desodiated state, respectively, generated by training a classification model;
performing, by the data sampling unit, oversampling and undersampling sequentially to solve data imbalance between the stable and unstable candidate materials in the pristine and desodiated states, respectively; and
deriving, by the selection unit, the stable material by selecting the candidate material stable in both the pristine and desodiated states.
